(19) United States
(12) Patent Application Publication
(10) Pub. No.: US 2023/0009804 A1
(43) Pub. Date: Jan. 12, 2023

(54) MACHINE-LEARNING TECHNIQUES FOR GENERATING ENTITY INSTRUCTIONS

(71) Applicant: Ateb, Inc., Raleigh, NC (US)

(72) Inventors: Robbert J. Ayshford, Raleigh, NC (US); James F. Boericke, JR., Raleigh, NC (US); Gerald Stephen Few, Raleigh, NC (US); Deborah N. Sheppard, Marco Island, FL (US); Frank P. Sheppard, Marco Island, FL (US)

(21) Appl. No.: 17/831,772

(22) Filed: Jun. 3, 2022

Related U.S. Application Data
(63) Continuation-in-part of application No. 14/466,474, filed on Aug. 22, 2014, now abandoned.
(60) Provisional application No. 61/869,330, filed on Aug. 23, 2013.
(60) Provisional application No. 61/979,220, filed on Apr. 14, 2014.

(51) Int. Cl. G16H 20/10; G16H 80/00; G06N 20/00

(52) U.S. Cl. CPC G16H 20/10; G16H 80/00; G06N 20/00

(57) ABSTRACT

A method for training and using a machine-learning model to determine an execution date for an instruction and to determine an associated action item. A machine-learning model can be trained by receiving entity data that includes historical prescription data, by generating training data by labeling data in the entity data, and by training the machine-learning model by mapping the labeled data to possible predictions for subsequent prescription executions for the entity. Data, which includes at least a prescription and a previous execution date can be received. A subsequent execution date and an associated action item can be determined. A prescription can be executed on the subsequent execution date. The action item can be executed before the subsequent execution date.

### FIELD OF THE INVENTION

The present invention relates generally to machine-learning techniques, and more specifically, but not necessarily exclusively, to machine-learning techniques for automatically determining periodic instruction execution dates and related information.

### BACKGROUND

Systems, methods and/or computer program products are widely used to run a pharmacy such as a stand-alone pharmacy and/or a pharmacy department of a larger entity. The systems may include one or more networked computers that execute management operations of the pharmacy, which may involve performing various tasks that may be related to improving compliance with one or more programs. Some of these tasks may involve receiving large amounts of input data, which may introduce significant complexities for existing systems.

### SUMMARY OF EMBODIMENTS OF THE INVENTION

A method can be used for training and using a machine-learning model to determine an execution date for an instruction and to determine an associated action item. A machine-learning model can be trained by receiving entity data that includes historical prescription data, by generating training data by labeling data in the entity data, and by training the machine-learning model by mapping the labeled data to possible predictions for subsequent prescription executions for the entity. Data, which includes at least a prescription and a previous execution date can be received. A subsequent execution date and an associated action item can be determined. A prescription can be executed on the subsequent execution date. The action item can be executed before the subsequent execution date.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a block diagram of methods, systems and computer program products according to some embodiments.

FIGS. 2-4 are flowcharts illustrating operations according to some embodiments.

FIG. 5 is a schematic diagram of scoring criteria according to some embodiments.

FIGS. 6-7 are flowcharts illustrating operations according to some embodiments.

FIGS. 8-12 are screenshots illustrating methods, systems and computer program products according to some embodiments.

FIG. 13 is a flow diagram illustrating operations according to some embodiments.

FIG. 14 is a diagram of one example of a machine-learning model according to some embodiments.

### DETAILED DESCRIPTION OF EMBODIMENTS OF THE INVENTION

The present invention now will be described more fully hereinafter with reference to the accompanying figures, in which embodiments of the invention are shown. This invention may, however, be embodied in many alternate forms and should not be construed as limited to the embodiments set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention as defined by the claims. Like numbers refer to like elements throughout the description of the figures.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including” when used in this specification, specify the presence of stated features, integers, steps, operations, elements, and/or components, but do not preclude the presence or addition of one or more other features, integers, steps, operations, elements, components, and/or groups thereof. As used herein the term “and/or” includes any and all combinations of one or more of the associated listed items and may be abbreviated as “/”. It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

The present invention is described below with reference to block diagrams and/or flowchart illustrations of methods, apparatus (systems) and/or computer program products according to embodiments of the invention. It is understood that a block of the block diagrams and/or flowchart illustrations, and combinations of blocks in the block diagrams and/or flowchart illustrations, can be implemented by computer program instructions. These computer program instructions may be provided to a processor of a special purpose computer and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, the present invention may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block may be separated into multiple blocks and/or the functionality of two or more blocks may be at least partially integrated.

Certain aspects and features of the present disclosure relate to techniques for determining execution dates for instructions and associated action items associated with an entity. Execution dates can include dates on which an instruction is executed by or on behalf of the entity, which can include an individual or other suitable entity. The associated action items can include notifications, reminders, and other action items that are transmitted to the entity to increase (compared to not using the action items) a likelihood that the instruction is executed on or by the execution dates. An instruction can include a prescription for medication, an instruction to communicate with another networked computing device, etc. For example, the entity can include a patient, and the instruction is a prescription issued to the patient by a provider.

In some examples, the instruction includes periodic execution such as executing the instruction once per day, once per week, once per month, or any other suitable periodicity. Executing the instruction accurately over the periodicity of the instruction can optimize an effectiveness of the instruction. For example, taking medication precisely at the time instructed to do so increases an effectiveness of the medication. Conversely, missing one or more instances of the instruction can decrease a maximum effectiveness of the instruction. For example, missing a dose of the medication reduces the effectiveness of the medication. Accordingly, ensuring that the entity precisely follows the periodicity of the instruction optimizes the effectiveness of the instruction. Additionally, determining the execution dates for the instruction and associated action items can improve a likelihood that the entity follows the instruction.

In some examples, machine-learning techniques are used to output predictions with respect to the techniques described herein. For example, the machine-learning techniques can output predictions for, or otherwise determine, the execution dates and the associated action dates. Additionally or alternatively, the machine-learning techniques can be used to predict whether the entity may benefit from the action items, etc. The machine-learning techniques can involve a machine-learning model that is trained using a set of training data that includes historical data relating to the entity. The historical data includes various instructions associated with the entity and associated data that indicates whether the entity followed the instructions, the respective execution dates, and action items taken with respect to the instructions and the respective execution dates. The historical data can be labeled to indicate an effect of respective data (e.g., action items, execution dates, etc.) on the likelihood that the instruction was executed properly. The labeled, historical data is used to train the machine-learning model to output, among other suitable outputs, predictions relating to entities, execution dates, and/or associated action items for an instruction.

In some examples, the machine-learning model is a neural network such as a convolutional neural network, a deep learning neural network, a recurrent neural network, or other suitable variations of neural networks. Alternatively, the machine-learning model includes other suitable types of models such as support vector machines, and the like. In other alternative examples, the machine-learning model can include other suitable types of artificial intelligence algorithms for outputting predictions relating to the execution date and associated action items of an instruction.

In some examples, the machine-learning techniques described herein may improve the functioning of a computing device, improve a technical field, be directed to a practical application, or any combination of these. For example, by periodically executing an instruction, fewer computing resources may be required to execute the instruction since a computing device need only request or receive initial input for executing the periodic instruction (e.g., instead of receiving continuous or periodic input). Additionally, compliance can be improved by determining action items associated with the instruction. While described with respect to compliance with medication, the techniques described herein can additionally improve compliance in other technical fields such as information technology, network security, and the like.

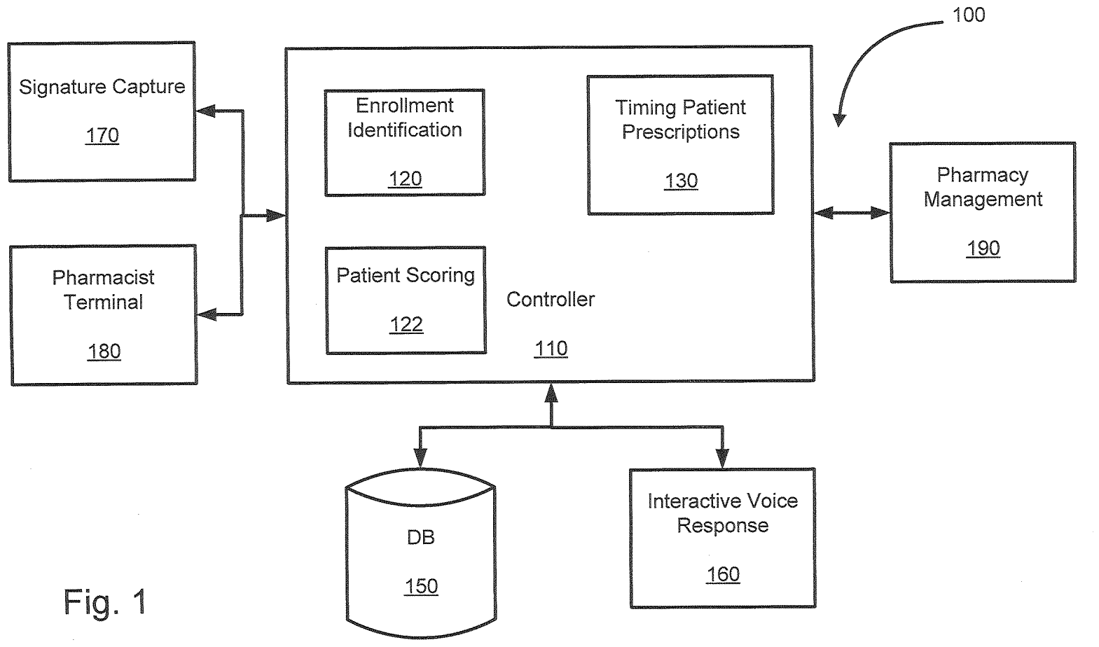

FIG. 1 is a block diagram of systems, methods and/or computer program products for controlling instructions according to some embodiments of the invention. In some examples, controlling the instructions involves a pharmacy system 100. As shown in FIG. 1, the pharmacy system 100 includes a controller 110 having an enrollment identification module 120, a patient scoring module 122, and a timing patient prescriptions module 130. In some examples, the patient scoring module 122 includes one or more machine-learning models or is otherwise capable of performing machine-learning techniques. The controller 110 may by in communication with database 150, an Interactive Voice Response (IVR) System 160, a signature capture device 170 and a pharmacist terminal 180 and/or other pharmacy management modules 190. The controller 110 may be embodied as one or more enterprise, application, personal and/or pervasive computer systems which may be connected by a network such as a local area network and/or a wide area network including the Internet. The controller 110 can coordinate interaction among the other components of FIG. 1. It will be understood that the functionality of the controller 110 can be centralized and/or distributed among the other components.

The IVR system components 160 may be coupled to one or more telephone lines to receive telephone calls from callers. The IVR system 160 can include prerecorded voice prompts such as prerecorded human voice segments, stored text-to-speech generated segments, text-to-speech segments that are generated on the fly, and/or use other conventional techniques for generating voice prompts. The pharmacy management module 190 may include computer systems and modules to manage patient records, manage doctor records, manage medication data, facilitate prescription fulfillment and/or perform other functions. Other pharmacy management systems 190 may be used to perform other pharmacy management functions.

The design and operation of the IVR system 160 and other pharmacy management systems 190 are well known to those having skill in the art and need not be described further herein. Moreover, it will be understood that the IVR system 160 and/or the other pharmacy management systems 190 may be combined to run on a single enterprise, application and/or personal computer system. Alternatively, these systems may be distributed over more than one enterprise, application, personal and/or pervasive computer systems which may be connected by a network such as a local network and/or a wide area network including the Internet.

Still referring to FIG. 1, the pharmacist terminal 180 may be used by a pharmacist to perform pharmacist functions in the pharmacy. For example, a barcode scanner also may be included and may be used by the pharmacist to identify a pharmaceutical prescription by scanning a barcode on a container (a bag, box, bottle, etc.) that corresponds to the pharmaceutical prescription. The signature capture system 170 may include one or more touch screen displays that are configured to accept a signature using a stylus and/or other device and may also include one or more keys and/or buttons (fixed and/or programmable) that may be activated by a user, for example, using a stylus and/or finger, to provide various user inputs. Various sequences of display screens may be displayed and user inputs may be accepted to provide prompt/response and/or information to a user of the signature capture system. The design and operation of a pharmacist terminal 180, a barcode scanner (not shown), and the signature capture device 170 are well-known to those having skill in the art and need not be described further herein.

The enrollment identification module 120, patient scoring module 122 and/or timing patient prescriptions module 130 are provided according to some embodiments of the present invention. The enrollment identification module 120, patient scoring module 122 and/or timing patient prescriptions module 130 may comprise hardware and/or software, which may include machine-learning or other suitable artificial models, algorithms, and/or software. It will be understood by those having skill in the art that the enrollment identification module 120, patient scoring module 122, the timing patient prescriptions module 130 and/or the database 150 may be integrated within one or more of the other components of the pharmacy system 100, in some embodiments. In other embodiments, the enrollment identification module 120, patient scoring module 122, the timing patient prescriptions module 130 and/or the database 150 may be provided on one or more enterprise, application, personal and/or pervasive computer systems that may be connected to one another using a network such as a local area network and/or a wide area network including the Internet. It will be understood by those having skill in the art that the term “database” is used herein to generically represent any kind of querying system, such as a rules engine, table, neural network, etc.

Systems, methods and/or computer program products according to embodiments of the present invention can provide an ability to determine automatic periodic execution dates for at least one instruction. For example, systems, methods, and/or computer program products can provide the ability to calculate automatic, periodic fill dates for one or more pharmaceutical prescription. Other suitable examples are possible. In some embodiments, systems, methods and/or computer program products align fill dates of one or more prescriptions such that the fill dates periodically occur at the same time. In some embodiments, systems, methods and/or computer program products determine a value for an entity in response to instruction and/or entity data associated with the entity to provide an instruction timing benefit value. For example, systems, methods, and/or computer program products can determine a value for a patient in response to prescription and/or patient data associated with the patient to provide a prescription timing benefit value. The instruction timing benefit value can be an estimate of a degree to which the entity would increase instruction compliance when instruction execution dates of one or more instructions are automatically periodically executed. When the instruction timing benefit value satisfies a predetermined threshold value, the one or more instructions may be automatically periodically refilled, e.g., to increase entity compliance. In some embodiments, the pharmacy system 100 may have access to specific entity data through the pharmacy management module 190 and/or one or more databases 150. Using this data, the enrollment identification module 120 and/or the patient scoring module 122 may identify whether an entity would benefit from automatically periodically executed instructions. In some embodiments, the timing benefit value allows a provider (e.g., a pharmacy) to focus efforts on entities (e.g., patients) that may be likely to increase compliance if they are offered enrollment in a program that automatically periodically executes associated instructions. For example, a patient may increase compliance with prescriptions if they are offered enrollment in the program that automatically periodically fills their prescriptions. The provider enrolls the entity in a program that periodically executes associated instructions (e.g., a patient's prescription and/or the prescriptions of other members of their household), for example, by receiving information about the entity and their associated instructions and calculating periodic execution dates as described herein.

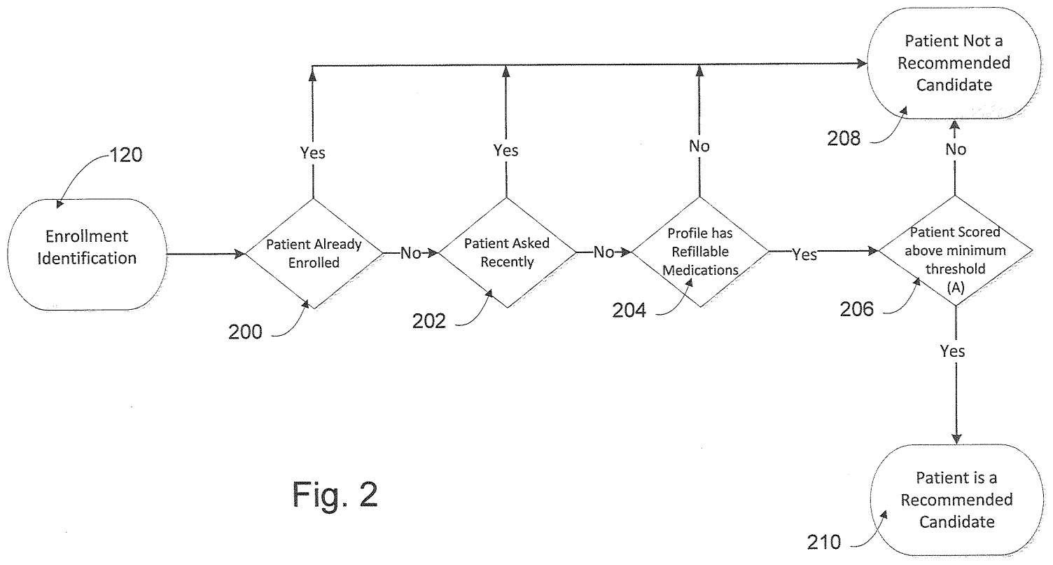

As illustrated and described, FIGS. 2-4 relate to enrolling a patient in a program that determines automatic medication prescription fill dates and determining a patient value, but other suitable examples of enrolling the entity in the program that determines automatic execution dates and determining an entity value are possible.

FIG. 2 is a flowchart of operations that may be performed to enroll an entity in a program that determines automatic execution dates. As illustrated in FIG. 2, the enrollment identification module 120 can determine whether a patient is a recommended candidate for enrollment to automatically periodically refill prescriptions. The enrollment identification module 120 makes various threshold queries, such as whether the patient is already enrolled (Block 200), whether the patient has been asked to enroll recently (Block 202) and whether the patient's profile has refillable medications (Block 204). If the patient is already enrolled (Block 200), or the patient has been asked to enroll recently and declined (Block 202), or the patient profile has no refillable medications (Block 204), then the patient is identified as not a recommended candidate for enrollment (Block 208). If the patient is not already enrolled (Block 200), the patient has not been asked to enroll recently (Block 202), and the patient profile has refillable medications (Block 204), then a value for the patient is determined or the patient is rated by the patient scoring module 122 (Block 206). If the patient is valued at a predetermined level (Block 206), such as above a minimum threshold, then the patient is identified as a recommended candidate for enrollment (Block 210). If the patient is not valued at a predetermined level (Block 206), then the patient is identified as not being a recommended candidate for enrollment (Block 208).

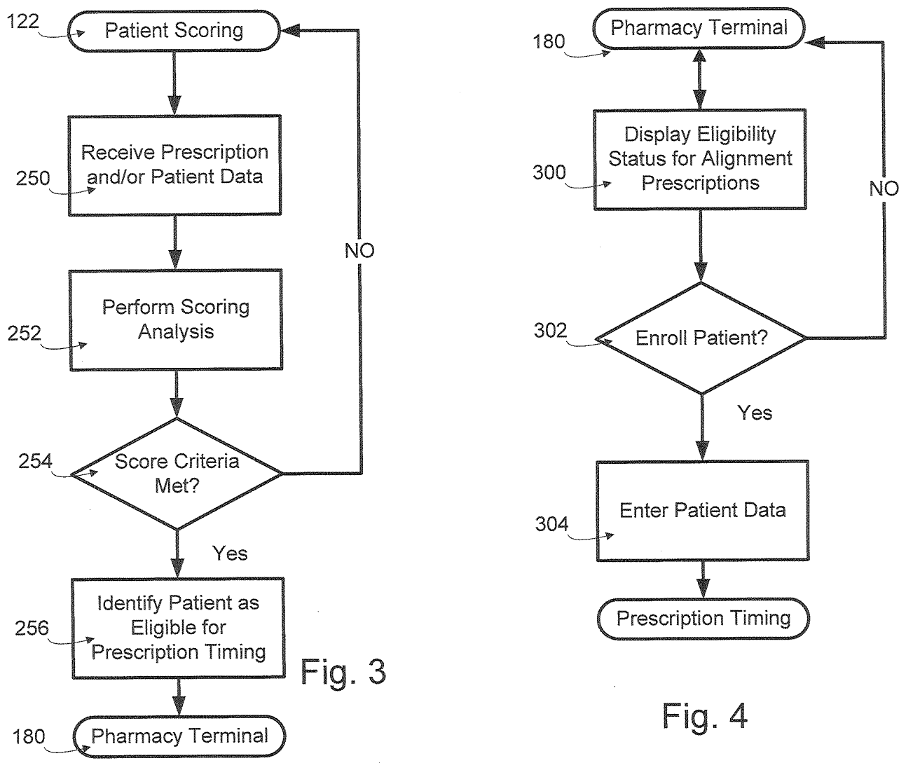

The patient value that may be calculated can be an estimate of a degree to which the patient would increase prescription compliance when prescription fill dates of one or more prescriptions are automatically periodically refilled, e.g., due to enrollment in a refill and/or reminder protocol. With reference to FIG. 3, the patient scoring module 122 can receive prescription and/or patient data (Block 250). A scoring analysis can be performed in response to the prescription and/or patient data (Block 252). If the value criteria is met (Block 254), the patient is identified as eligible or recommended for a prescription timing or periodic refill protocol (Block 256). If the value criteria is not met (Block 254), then the patient is not identified as eligible, and in some embodiments, additional patients and/or patient data may be analyzed. Although embodiments of the invention are described with respect to automatically periodically refilling prescriptions for a patient, it should be understood that the prescriptions that are automatically periodically refilled may relate to two or more patients, such as when the two or more patients are in the same household. In some embodiments, prescription records may be collated or collected and associated with a particular patient and/or related household. The operations of FIGS. 2 and 3 may be performed during a particular pharmacy transaction, such as when the patient is refilling a prescription and/or the operations of FIGS. 2 and 3 may be performed for one or more patients and/or patient records independent of a pharmacy transaction so that the pharmacist or pharmacy technicians may contact patients by any suitable technique, including direct phone calls, IVR phone calls, text messages, and the like.

With reference to FIGS. 3 and 4, if the patient is eligible for prescription refill timing alignment (Block 256), then the pharmacist terminal 180 displays the eligibility status, e.g., to the pharmacist or pharmacy technician (Block 300). The patient is provided with an opportunity to enroll in the periodic refill protocol (Block 302). If the patient agrees to enroll (Block 302), then the patient data is entered into the refill timing protocol for follow up (Block 304). In some embodiments, the patient consent is recorded in order to comply with patient confidentiality requirements, such as the Health Insurance Portability and Accountability Act (HIPAA).

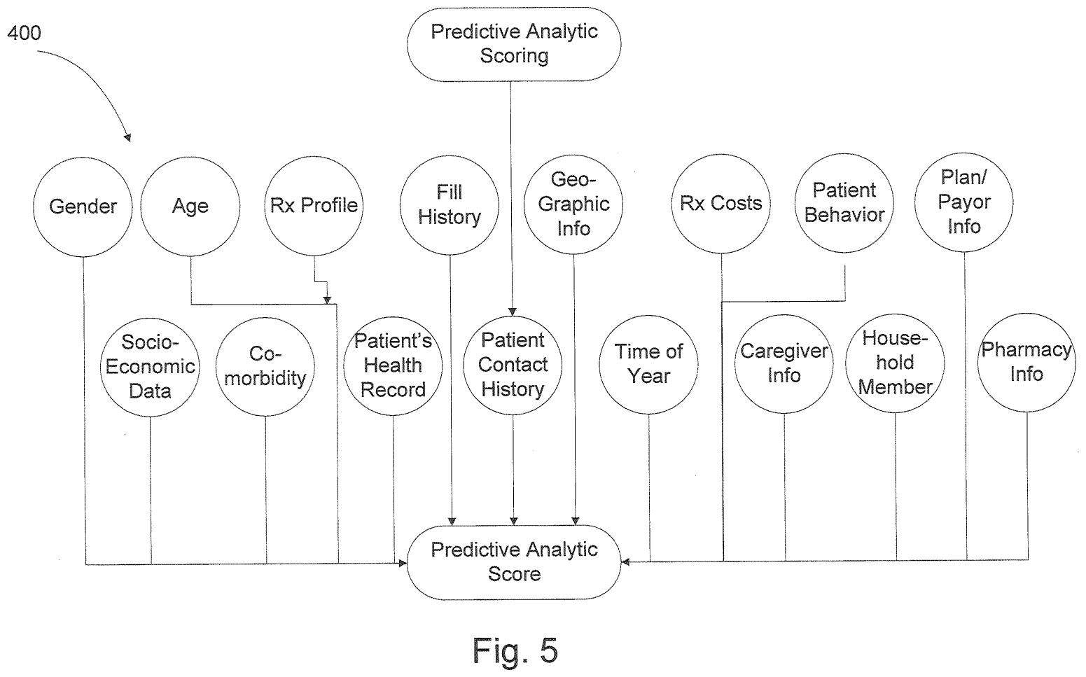

As illustrated in FIG. 5, scoring an entity to estimate a degree to which the patient would increase prescription compliance if the patient's prescriptions were automatically periodically refilled can include predictive analytic scoring. Predictive analytics may utilize a variety of techniques from statistics, modeling, machine learning and/or data mining to analyze current and/or historical data to forecast or make predictive valuations about future events, such as a degree to which an entity may increase instruction compliance when enrolled in an instruction timing alignment protocol. The predictive analytic value may be determined by predetermined criteria 400 including, in an example in which the entity is a patient, the patient's gender, age, prescription profile (prescription data, including the type of prescription), the prescription filling history, the patient's geographic information, the prescription costs, the patient's behavior, the patient's payor or insurance information, socio-economic data, health data (including the co-morbidity of other conditions), the patient's health records, the contact history of the patient by the provider or other health care professionals, the time of year, the patient's caregiver information, an identification of other members of the patient's household and/or information about the provider where the patient fills prescriptions. The entity's behavior can include the entity's past instruction history with respect to timeliness, including the number of days late to execution, consistency of being late, the duration of lapses in executions, the percentage of on-time instruction executions, the likelihood that the entity will pick up a certain type of medication as compared to other types of medication (including the behavior of picking up multiple medications when certain other medications are due or overdue), a responsiveness to other initiatives (e.g., patients that perform well in an automatic refill program may be good candidates for medication alignment), and the entity's pattern(s) of executing instructions such as the time of the month (beginning, middle or end) or days of the week. Regression models to establish a mathematical equation that represent statistical interactions between different variables may be used as would be understood by those of skill in the art. Regression models include linear regression models (multivariate regression), discrete choice models, logistic regression models, multinomial logistic regression models, probit regression models, time series models, survival analysis or time to event analysis, classification and regression trees, multivariate adaptive regression splines (MARS). Machine-learning techniques can include those techniques known to those of skill in the art, including neural networks, multilayer perceptron, radial basis functions, support vector machines, naïve Bayes conditional probability rule, nearest neighbor algorithms, geo-spatial predictive modeling, and the like.

In some examples, a trained machine-learning model (e.g., a neural network) determines the predictive analytic value. For example, the machine-learning model is trained using training data that includes one or more types of historical data (e.g., similar to the predetermined criteria 400). Training the machine-learning model can involve tuning weights of the machine-learning model to optimize mapping between inputs (e.g., data relating to the entity) and outputs (e.g., the predictive analytic value). The trained machine-learning model can receive, as input, the predetermined criteria 400 and/or other suitable input data relating to the entity, and the trained machine-learning model can output the predictive analytic value for the entity.

In particular embodiments, the predictive analytic value that estimates a degree to which an entity may increase compliance when instruction execution dates are automatically periodically executed includes providing compliance benefit information for two or more entities. The compliance benefit information includes entity data and an indication of a change in instruction compliance when execution dates of one or more instructions are automatically periodically executed. A compliance benefit model may be created based the compliance benefit information for predicting which entities will increase compliance when the execution dates of one or more instructions are automatically periodically executed. The compliance benefit model can be created by regression analysis of the compliance benefit information. The instruction timing benefit value can be based on the compliance benefit model and is a prediction of a relative likelihood that an entity will increase instruction compliance when the execution dates of one or more instructions are automatically periodically executed.

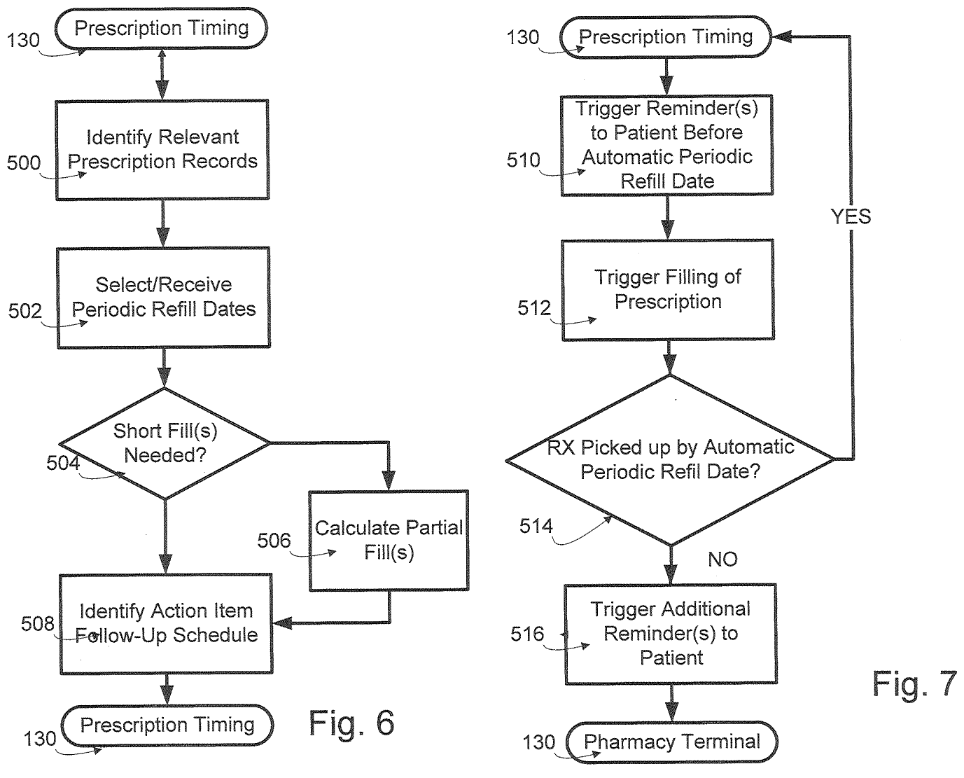

FIGS. 6 and 7 are described as examples of the techniques disclosed herein with respect to prescription timing and pharmacies. Other examples of instruction timing, providers, and the like are possible.

As illustrated in FIG. 6, the prescription timing module 130 can calculate automatic periodic fill dates for one or more prescriptions as follows. One or more prescription records associated with the patient and/or other household members of the patient are identified (Block 500). Automatic periodic fill dates are selected and/or received (such as received by a processor as an input) for the one or more prescription records (Block 502). Optionally, the prescription timing module 130 may calculate any short fills if the days supply of the prescription is less than or greater than a time period between a last fill date and a next fill date such as the automatic periodic fill date. A “short fill” generally refers to deliberately filling only part of a prescription without an intention to provide the remaining portion of the prescription. In contrast, a “partial fill” generally indicates that the pharmacy only had a limited inventory, and consequently, gave the patient an initial partial supply with the intent to give the patient the rest of the supply when the drug becomes available. For example, if the time period between the last fill date and the automatic periodic fill date is greater than the days supply of the prescription, then a short fill may be calculated and provided to the patient. In this case, the short fill would generally have a days supply equal to a difference between the days supply of the prescription and the time period between the last fill date and the first automatic fill date, and the short fill may be immediately provided to the patient, e.g., during the alignment process. If the time period between the last fill date and the automatic periodic fill date is less than the days supply of the prescription, then a short fill may be calculated that has a days supply generally equal to the number of days between the last fill date and the first automatic fill date. The short fill may be provided to the patient immediately during the alignment process or at a date between the last fill date and the first or second automatic fill dates. When a short fill of a prescription facilitates a selected periodic fill date (Block 504), the short fill(s) are calculated (Block 506). An action item or follow-up schedule is identified (Block 508). The action item(s) may include reminders before and/or after the automatic fill dates and may be provided to pharmacy staff by a computer system or automatically executed, for example, by one or more elements in the system described in FIG. 1.

In some examples, the prescription timing module 130 includes, or otherwise uses, machine-learning techniques to perform the operations of one or more of the block 502, the block 504, the block 506, and the block 508. For example the prescription timing module 130 can include a machine-learning model that can determine partial fills, short fills, prescription timings, and the like. The machine-learning techniques performed by the prescription timing module 130 may provide predictions for optimizing compliance by the patient or other entity with instructions or prescriptions associated with the entity.

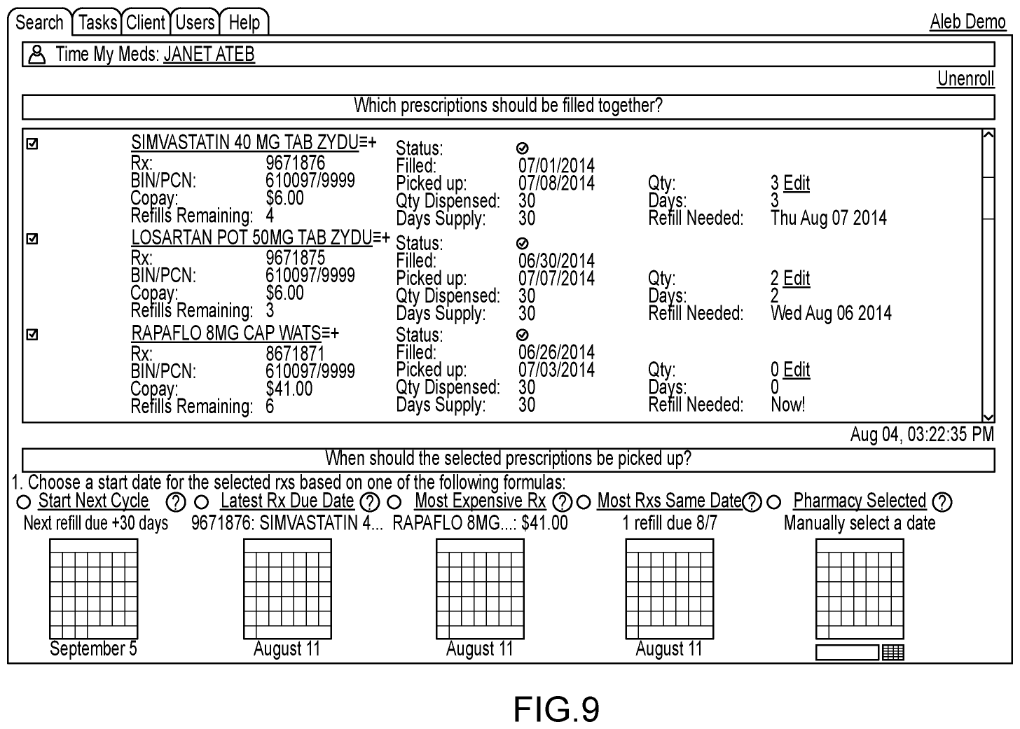

In some embodiments, the prescription records eligible for automatic periodic refills may be displayed, for example, as shown in FIG. 9. If a prescription meets certain predetermined criteria indicating that the prescription is a good candidate for automatic periodic refills, then the prescription may displayed with a recommendation that the prescription be selected for automatic periodic refills. However, the user may select the prescriptions that will be automatically and periodically refilled, and in some embodiments, the user may choose to select prescriptions for automatic and periodic refills independently of the recommendation. Therefore, the user may (or may not) select prescriptions that are recommended for automatic periodic refills, and the user may (or may not) select prescriptions that are not recommended for automatic periodic refills. Accordingly, the prescriptions that are automatically and periodically refilled may include both recommended and not recommended prescriptions. Prescriptions that are not recommended for periodic refill may include those that are not refillable and/or prescriptions that are taken on an “as needed” basis, such as allergy medication. In some embodiments, the prescriptions that are not refillable or not recommended for automatic refills may also be visually indicated on the display screen. Prescriptions that are not refillable may include, for example, controlled substances or antibiotics that are not refillable and may be ineligible for an automatic refill program or prescriptions for which the prescriber has not authorized any (or limited) refills.

The user may select an alignment period for the prescriptions and/or a start date for the first refill. As used herein, a “supply length” or “days supply” refers to a standard period or length of a prescription as prescribed by a prescribing health care provider. For example, a thirty day supply for a once a day pill would be thirty pills. If all of the prescriptions are for the same number of days supply, then the alignment period may be automatically set as the number of days supply from each selected medication. In particular embodiments, the user may select multiple prescriptions onto a single cycle. The cycle may have a minimum cycle length (e.g., 28 days). The prescription timing module 130 may display a warning if prescriptions whose cycle length is less than the cycle or up to a certain number of days greater than the cycle. In some embodiments, the prescription timing module 130 may use machine-learning techniques to determine whether to display the warning for certain entities or users. The user may choose to ignore the warning and continue to add the prescription for automatic refills, or the user may remove the prescription from selection based on the warning. Prescriptions with a supply length that is greater than the current cycle may be ineligible for grouping together for alignment and may be provided with their own cycle length. Multiple alignment cycles may be identified for a single patient or customer. If all medications have a supply length that is a multiple of other medications, then the shortest days supply may be selected for the alignment period. Those with a longer days supply are still set to a alignment period based on the relevant number of days supply, but may be set to align with a future alignment date. For example, if two medications with a thirty day supply, a medication with a sixty day supply, and a medication with a ninety day supply are selected, all four may be aligned such that the start date for all of the medications is the same. The medications with a thirty day supply are filled every thirty days, the medication with a sixty day supply is filled only at multiples of sixty days, and the medication with the ninety day supply is filled only at multiples of ninety days.

If there is a supply day mismatch between prescriptions, then the user may be visually alerted on the display screen. For example, if the user tries to align a prescription with a thirty day fill period and a prescription with a twenty-eight day fill period, then a warning may be displayed. The user may select whether to include the mismatched medications together and may also select the desired period. For example, the user may refill the twenty-eight day prescription every thirty days in order to align with the thirty day supply prescription.

The automatic periodic refill dates, however, may be based on any suitable factor or factors. In some embodiments, the automatic periodic refill dates may be based on minimizing the amount of partial refills needed to synchronize more than one prescription. The automatic periodic refill dates may also be based on other factors. For example, a pharmacy that delivers prescriptions directly to a patient may determine suggested refill dates based on the efficiency of delivery routes of the pharmacy to synchronize filling prescriptions from patients on a particular delivery route. Other factors include economic factors of the pharmacy such as filling prescriptions at a particular time of the month to facilitate reimbursements, and/or advising patients to avoid patient out-of-pocket expenses. Suggested refill dates may be selected in order to more effectively manage inventory, such as with respect to more expensive medications.

In some embodiments, the estimated costs to the patient for the prescriptions at the next synchronization date may be calculated after the medications and corresponding alignment periods are selected by the user. The user may be given the option to change the synchronization dates, for example, to spread the cost of the prescriptions over a longer period of time by having two or more off-set synchronization dates. In some embodiments, the estimated costs to the patient for the prescriptions may be used to calculate and provide suggested alignment dates. For example, a refill date corresponding to a prescription with a highest copay of a group of prescriptions may be provided or suggested to the user, which may reduce the costs of short fills.

In some embodiments, an alignment date may be suggested to the user that corresponds to a latest refill date corresponding to a prescription with a refill date that is a latest date of a group of prescriptions. For example, if the current date is Day 0, and the patient has three 30-day prescriptions A, B and C that were last fill on Day −20 (Prescription A), Day −15 (Prescription B), and Day −1 (Prescription C). Then, the prescriptions are each due for a refill on Day 10 (Prescription A), Day 15 (Prescription B) and Day 29 (Prescription C). The last refill date (Day 29) may be selected as a recommended first aligned refill date to refill all of the prescriptions. Prescription A would need a partial refill of 19 days and Prescription B would need a partial refill of 14 days in order to provide the patient with a continuous supply of the prescription.

As another example, an alignment date may be calculated by determining on which day the most prescriptions are due in order to minimize the number of short fills required to get all the prescriptions aligned. For example, five 30-day prescriptions are selected A (due on the 22nd), B, C, D (due on the 28^{th}), and E due on the 5^{th} of the following month). The alignment date would be selected as the 28^{th} because three prescriptions all come due on that date. A further variant of this date is to select a neutral date that aligns with none of the existing prescriptions but would allow the pharmacy to fill the most number of prescriptions without having to perform short fills. In the immediately preceding example, the pharmacy could select an alignment date of the 23^{rd} and process full fills of medications A, B, C, and D and only have to fill a short fill of prescription E. This is because pharmacies are generally allowed to fill prescriptions up to a predetermined number of days early (typically five days early) for patients. Hence, a date that reduces the need for short fills across all selected prescriptions may include filling some prescriptions a day or two late and others up to 5 days early.

As another example, the prescriptions and available alignment dates may be reviewed to reduce the total cost to the patient by reducing or eliminating as many short fills as possible and/or finding the cheapest short fills. For example, many pharmacies give away or sell very inexpensively many generic medications. One or more suggested alignment date(s) may be selected such that across all medications, the co-pay associated with short fills are reduced or minimized, for example, by selecting an alignment date that generally coincides with the next refill date of prescriptions for which short fills are expensive while allowing short fills for prescriptions that may be inexpensive to short fill in order to reduce or eliminate the cost of short fills.

As another example, the suggested alignment day may be selected based on pharmacy activity. For example, a pharmacy routinely does diabetes workshops during the second week of each month. For patients with one or more diabetes medications being aligned, the pharmacy may select an alignment date that corresponds with the monthly diabetes workshops.

Once the pharmacist finalizes the prescriptions to synchronize and has selected the synchronization period, a synchronization start date may be selected for filling all of the prescriptions on the same synchronization period. In some embodiments, a default start date may be selected, for example, based on the first available date that the prescriptions could be posted or the date by which all of the selected medications would be finished based on the supply length of the prescriptions.

In some embodiments, the prescription timing module 130 may track two aspects of the prescription number of days supply: 1) the number of days supply for which the prescription was last filled, and 2) the remaining number of days supply, which may be based on either a number entered by a user or a calculated number of days supply remaining that is calculated based on the sold date of each prescription and the current date. In some embodiments, the default synchronization start date is calculated as the earliest available date that permits a predetermined set of refill reminders or follow-up schedule.

The follow-up schedule can include one or more reminder action items for the pharmacy staff and/or computer system(s) to interact with the patient at dates before one of the automatic periodic refill date and/or a prescription fill trigger at a date before the automatic periodic refill date. As illustrated in FIG. 7, the prescription timing module 130 can trigger reminders to a patient before the automatic periodic refill date (Block 510). The reminders may be sent to the patient by various media, including voice messages via the IVR system 160 (FIG. 1), electronic messages (text messages on a mobile device, email messages and the like) and/or in-person telephone calls made by a pharmacist or other pharmacy employee. The prescription timing module 130 can also trigger the filling of the prescription (Block 512), for example, a predetermined number of days before the calculated synchronization date so that the prescription is automatically refilled without requiring that the patient contact the pharmacy directly. If the prescription is picked up by the calculated automatic periodic refill date (Block 514), then the prescription timing module 130 proceeds to the next prescription cycle, such as the next thirty day cycle for thirty day prescriptions. If the prescription is not picked up by the patient by the automatic periodic refill date or a predetermined date after the automatic periodic refill date (Block 514), then additional reminders may be triggered to the patient (Block 516). In some embodiments, the prescription timing module 130 uses machine-learning techniques to trigger action items (e.g., determining an alignment date, automatic instruction execution and/or prescription filling, reminders and/or additional reminders, and the like).

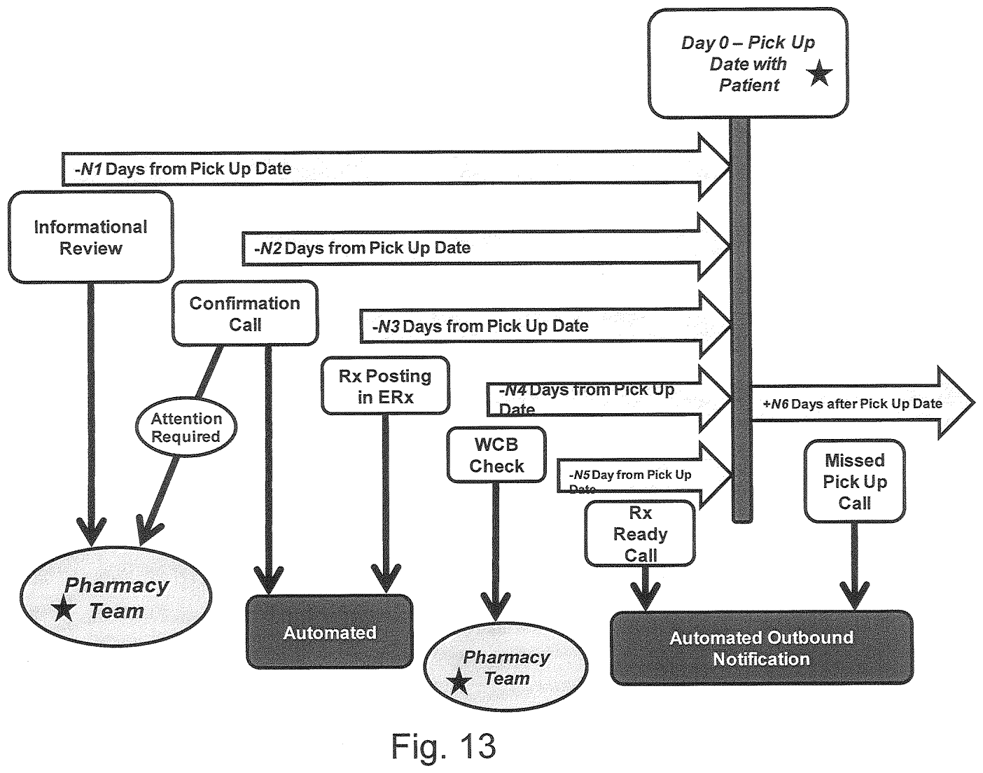

An exemplary follow-up schedule of possible action items is illustrated in FIG. 13. As illustrated in FIG. 13, at N1 days from the synchronization date or pick-up date, the prescription timing module 130 issues a reminder to the pharmacy team to have an informational review of the prescriptions. The informational review allows the pharmacy to determine if discussions with the patient should occur. For example, the pharmacist may note that the patient is taking a prescription that is designated as a “high risk” medication, which may be replaced with a lower risk medication. The pharmacy may also note that the patient is missing a medication that would usually be taken or indicated for use by the medications that the patient is taking. Accordingly, the informational review may include a manual review of the patient's records so that the pharmacy team may proactively manage the patient and/or identify any appropriate health interventions. At N2 days before the pick-up date, a confirmation call to the patient is initiated either by an automatic call or by reminding the pharmacy team to call the patient. The confirmation call may include the pharmacy asking the patient if anything has changed since their last alignment or pick up date. If changes are identified, the pharmacy has an opportunity to make those changes so that any changes or modifications are in order when the patient receives his or her medication (in some embodiments, about a week after the confirmation call). At N3 days before the pick-up date, the prescription is posted as ready for the pharmacy team to automatically refill the prescription. At N4 days before the pick-up date, a reminder is sent to the pharmacy team to verify that the prescription has been filled or an automated call is delivered, and at N5 days from the pick-up date, a call is made to the patient that the prescription is ready. If the prescription is not picked up by the patient by N6 number of days past the pick-up date, then additional calls are made either by an automated calling service or by the pharmacy team.

Although embodiments according to the present invention are described with respect to scoring and/or identifying a patient for enrollment to automatically refill prescriptions, it should be understood that one or more prescriptions may be aligned without necessarily scoring/identifying the patient, for example, as described in FIGS. 3 and 4.

In some embodiments, the operations of FIGS. 6 and 7 may be repeated to set up periodic automatic refills for a plurality of patients. The patients may optionally be identified as described with respect to FIGS. 3 and 4. The action items, such as the reminder triggers discussed with respect to FIG. 7, may be provided to the pharmacy staff and/or via computer implemented methods periodically. For example, a computer system may generate a list of all of the patients who have outstanding action items associated with them once or twice (or more) times a day or a number of times per week.

In some embodiments, the recurring dates of an automatic periodic refill date includes two or more dates, each of the synchronization dates being associated with a period refill of the two or more prescriptions. Thus, prescriptions with different refill periods (e.g., 28 days, 30 days and 90 days are typical refill periods) may be synchronized to the same or different automatic periodic refill dates. In particular embodiments, the prescriptions with different refill periods are not synchronized with one another. For example, all of the refills having a 30 day refill period may be synchronized with one another, and all of the refills with a 90 day refill period are synchronized with one another but are not synchronized with the 30 day refill period prescriptions. The automatic periodic refill date can be a recurring calendar date, e.g., a date that is based on a time period in the calendar such as the first Monday of the month, to generally encourage the patient to remember to pick up his or her prescriptions at a particular recurring time of the month. In particular embodiments, the time period in the calendar that is set as the automatic periodic refill date does not change even if the patient does not refill the prescription by the calculated refill date.

Without wishing to be bound by any particular theory, by automatically periodically refilling the patient's prescription, medication compliance may be increased. The patient may become accustomed to getting his or her prescriptions refilled, for example, at a certain time of the month without needing to contact the pharmacy to initiate the refill. In some embodiments, the pharmacy is proactively reaching out to the patient and managing the patient's care, such as by suggesting health interventions or alternative medications, instead of relying on the patient to initiate a refill. The pharmacy/patient interaction may create an accountability or incentive to drive adherence to the refill schedule and better medication compliance. This behavior may be understood according to a transtheoretical change behavior model (precontemplation, contemplation, preparation, action and maintenance). The automation of the pharmacy team action items or reminders to complete the action items may allow the alignment techniques to be performed for a relatively large number of patients and/or to proactively manage patients. Moreover, using predictive analytics, a value can be determined for patients indicating whether their compliance is likely to increase when their prescription(s) are periodically and automatically refilled.

In particular embodiments, the action items, such as the reminder triggers and refill triggers discussed with respect to FIG. 7, may be based on customized or semi-customized characteristics of the patient. For example, a patient who has had very poor compliance with refilling medication may receive more reminders than a patient who has had better compliance. Moreover, additional event triggers may be used, such as a prompt for the pharmacist to provide information to the patient, e.g., to encourage medication compliance and/or advise the patient as to the consequences of noncompliance.

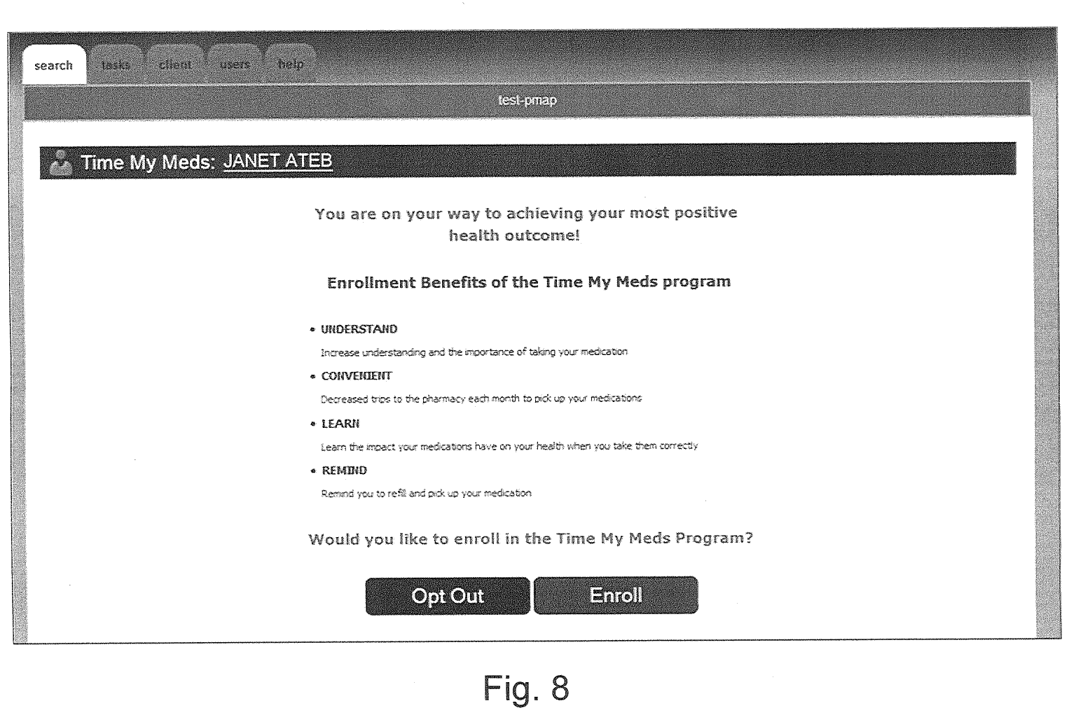

As shown in FIG. 8, when a patient has been identified for enrollment, for example, as illustrated in FIG. 2, the pharmacy terminal may display an enrollment screen. As illustrated, the enrollment screen provides information about the periodic refill protocols including a list of benefits to the patient. The patient then has the option of either enrolling in the periodic refill protocol program or opting out. As illustrated in FIG. 9, if the patient has opted to enroll in the refill protocol, the pharmacist can select which prescriptions associated with the patient and/or the patient together with other members of the patient's household should be included in the periodic refill program. For example, the pharmacist can select all of the prescriptions with the same refill period (e.g., all of the 30 day prescriptions) to be in the periodic refill program.

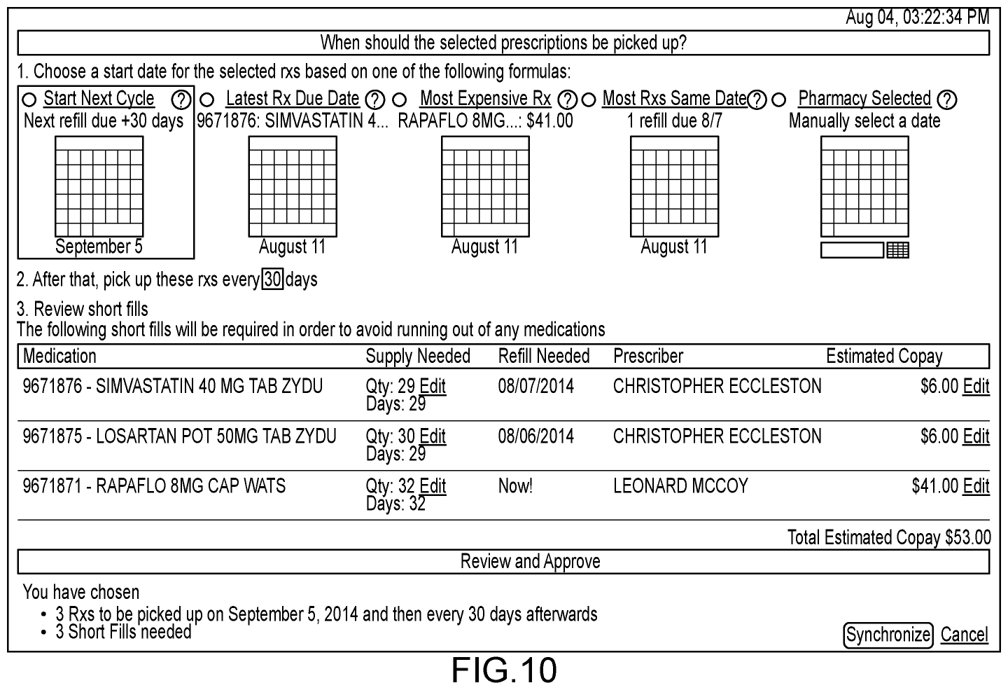

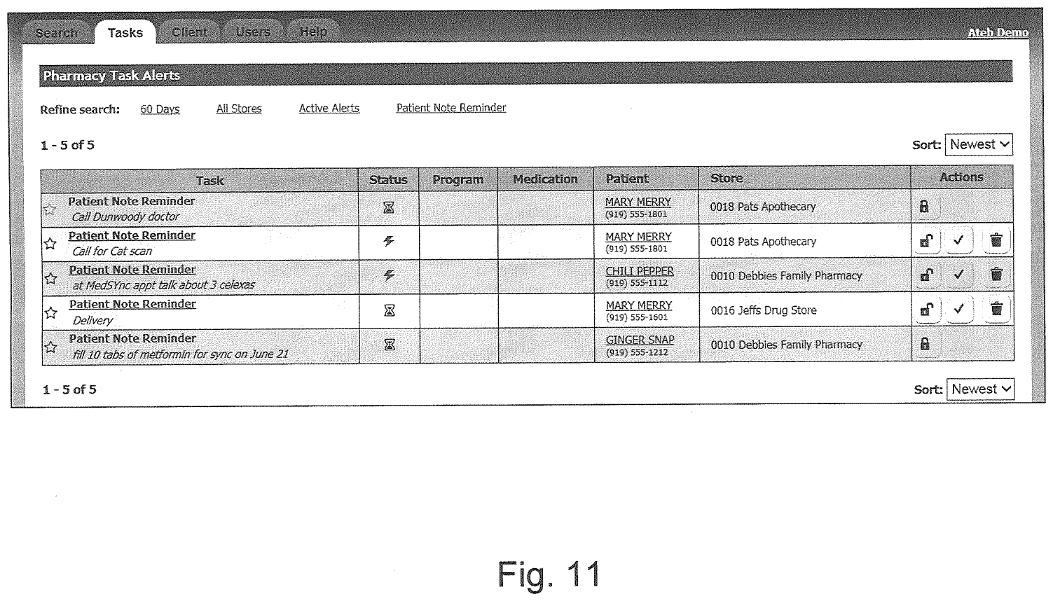

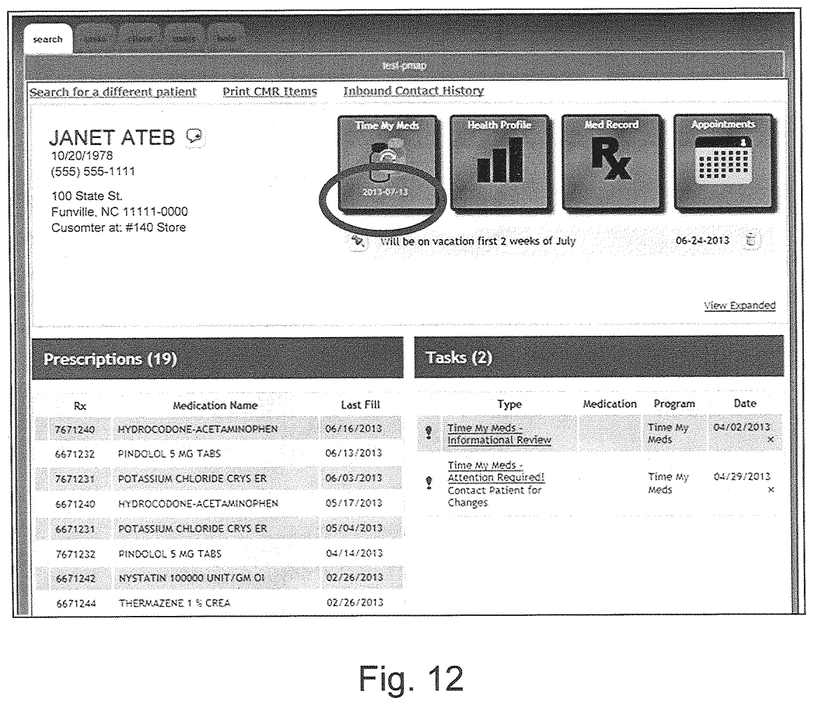

As shown in FIG. 10, the pharmacist can review the prescriptions and then select an icon to synchronize the prescriptions. As illustrated in FIG. 11, the pharmacy terminal displays all of the synchronized prescriptions and the proposed synchronization or automatic periodic refill date for confirmation by the pharmacist and/or patient. In some embodiments, the patient can modify the refill dates, e.g., if the patient prefers a different time or periodic date. As shown in FIG. 12, the pharmacy terminal then displays the pharmacy information regarding the automatic periodic refill, e.g., including the next refill date, the prescription information, and the task(s) for the pharmacy, such as when to provide the patient with reminders and/or when to fill the prescription. Although embodiments according to the invention are described in FIGS. 8-12 as displaying information on a pharmacy terminal, it should be understood that the same or similar information and options may be displayed to the pharmacy patient directly on a home computer or portable electronic device, such as a smart phone.

In some embodiments, the prescription timing module 130 may also provide the pharmacy team with information to review with the patient, for example, about the benefits of taking his or her medication regularly and adhering to medication synchronization. The prescription timing module 130 may provide the pharmacy team with forms to confirm that the patient is choosing to enroll in a synchronization program. Worksheets may also be used to list short fills that the pharmacy may prepare in order for the patient to have access to medication until the first alignment start date. Information may also be sent to the doctor or other prescriber informing them of the benefits of the alignment program. In some embodiments, the prescription timing module 130 may also send the prescriber a refill authorization form that requests that the prescriber authorize a new prescription for the patient so that all of the alignment prescriptions expire at around the same time (for example, within a month).

In some embodiments, the alignment dates may or may not be the same for each of the alignment medications, which may provide additional flexibility in creating multiple alignment cycles for the same patient or household, for example, if a patient cannot afford to align all of the prescriptions at the same time or if the patient has medications that are not filled for the same number of days supply.

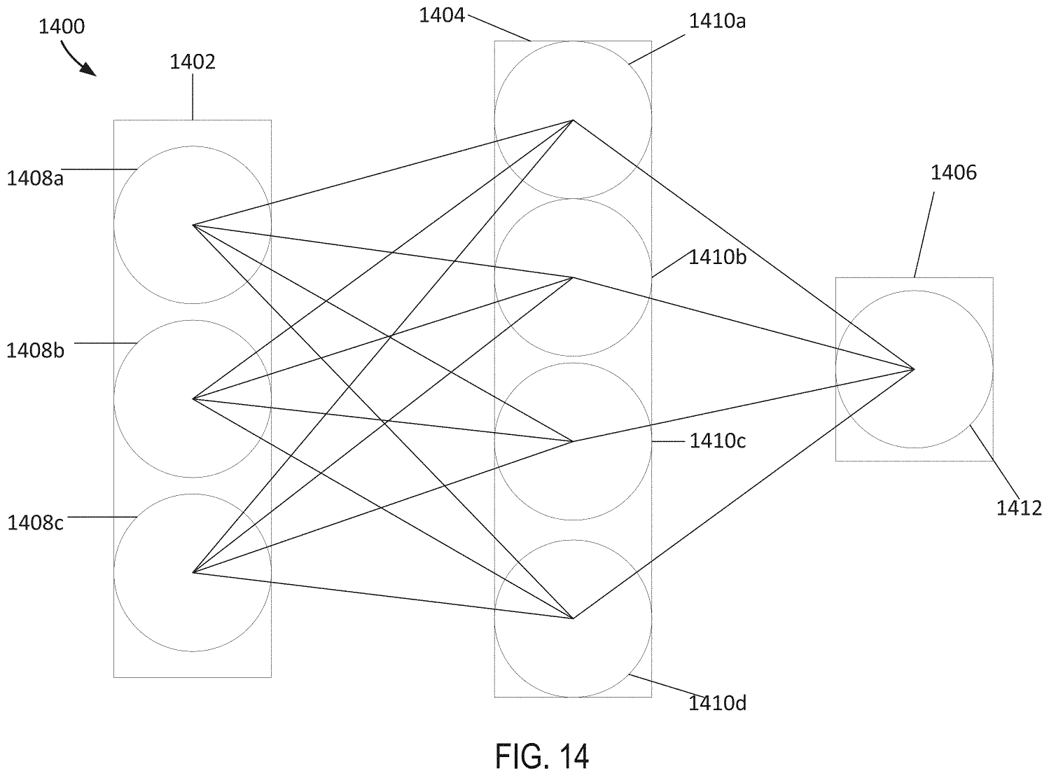

FIG. 14 is a diagram of one example of a machine-learning model 1400 that can be used to perform the techniques described herein. While illustrated as a neural network with a set of layers, the machine-learning model 1400 can alternatively be any other suitable type of machine-learning model or algorithm capable of performing the techniques described herein.

The machine-learning model 1400 includes an input layer 1402, one or more intermediate layers 1404, and an output layer 1406. The machine-learning model 1400 can include other suitable amounts and/or types of layers. The input layer 1402 includes inputs 1408a-c, which can include various input data for performing the techniques described herein. For example, the inputs 1408a-c can include historical instruction or prescription data, such as a type of instruction or prescription, whether the entity executed the associated instruction, the timing of the executed instructions, etc., for an entity or a patient.

The intermediate layers 1404 are illustrated as a single layer, but more than one (e.g., two, three, four, five, etc.) are possible. In some embodiments, the intermediate layers 1404 can include one or more hidden layers, one or more convolutional layers, or one or more other types of layers suitable for use in the machine-learning model 1400. The machine-learning model 1400 can map elements of the input layer 1402 to elements or data 1410a-d of the intermediate layers 1404. For example, the machine-learning model 1400 may include various weights that map one input of the input layer 1402 to one element of the intermediate layers 1404 (and also from the elements of the intermediate layers 1404 to the output 1412 of the output layer 1406). The weights may be tuned by training the machine-learning model 1400. Training the machine-learning model 1400 can involve tuning the weights by inputting labeled data (e.g., training data that includes labeled historical instruction or prescription data) into the machine-learning model 1400 and mapping the labeled inputs to expected or actual outputs. Other suitable techniques for training the machine-learning model 1400 are possible.

The output layer 1406 includes the output 1412. In some embodiments, the output 1412 can include a prediction, a binary selection, a list of selectable options, or any other suitable output from the machine-learning model 1400. For example, the machine-learning model 1400 can receive as input (e.g., via the input layer 1402) instruction or prescription data relating to an entity or patient. The machine-learning model 1400 may map or otherwise transform the input through the intermediate layers 1404 and generate the output 1412 at the output layer 1406. In this example, the output 1412 may include a patient value or entity value, a prediction of whether the entity or patient may increase instruction/prescription compliance by enrolling in the program described above, a selectable list of action items to take with respect to the entity or patient, or any other suitable output from the machine-learning model 1400. In some embodiments, the output 1412 can be used to determine execution dates, the associated action items, whether to enroll (or offer enrollment to) the entity, or in other suitable techniques as described herein.

In some embodiments, a method for periodically refilling one or more prescriptions for one or more patients is provided, each of the prescriptions having a days supply associated therewith. The method includes a) receiving one or more prescriptions to periodically refill, each of the one or more prescriptions having a next fill date associated therewith, wherein the next fill date is a date when the prescription was last filled incremented by a days supply for the prescription; b) receiving an alignment date selection on a processor for the one or more prescriptions; c) automatically triggering a refill of the one or more prescription on the alignment date; and d) triggering one or more action items on a processor before and/or after the alignment date.

In some embodiments, the one or more action items before and/or after the alignment date comprise at least one action item to inform the patient that the prescriptions are ready for pick up. The one or more action items may include at least one action item to confirm the refill before the alignment date. In some embodiments, the method further includes determining whether the patient has picked up the prescription within a predefined time period, and if the patient has not picked up the prescription within the predefined time period, the one or more action items comprises an action item to remind the patient that the refill pick up has been missed. In some embodiments, the method further includes repeating steps a)-d) for a plurality of patients, and periodically providing a list of the one or more action items for the plurality of patients. The one or more action items may be electronic reminders to a pharmacy staff to contact a patient. In some embodiments, the method may further include calculating one or more suggested alignment dates by determining the next fill dates for each of the one or more prescriptions and selecting the latest next fill date as a suggested alignment date.

In some embodiments, the method includes determining if any of the one or more prescriptions requires a short fill prior to the alignment date in order to supply the patient with medication until the alignment date; and if a short fill is required prior to the alignment date, calculating an amount of the short fill. The method may include filling the amount of the short fill and providing the amount of the short fill to the patient. The amount of the short fill may be provided to the patient when the patient selects the alignment date. In some embodiments, the method includes calculating a cost of the short fill prior to filling the amount of the short fill to the patient.

In some embodiments, the one or more prescriptions comprise a plurality of prescriptions, and, if a refill supply amount for one or more of the plurality of prescriptions is different, then the step of receiving an alignment date selection comprises receiving a different alignment date selection for each group of the plurality of prescriptions having the same refill supply amount. In some embodiments, the one or more prescriptions include a plurality of prescriptions, and at least one of the one or more suggested alignment dates include a refill date corresponding to a prescription with a highest copay of the plurality of prescription. The one or more prescriptions may include a plurality of prescriptions, and at least one of the one or more suggested alignment dates may include a latest refill date corresponding to a prescription with a refill date that is a latest date of the plurality of prescriptions.

In some embodiments, scoring a patient with a processor in response to prescription and/or patient data associated with the patient to provide a prescription timing benefit value, the prescription timing benefit value comprising an estimate of a degree to which the patient would increase prescription compliance when prescription fill dates of one or more prescriptions are periodically automatically refilled; and when the prescription timing benefit value satisfies a predetermined threshold value, performing steps a)-d).

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method comprising:
training, by a processing device, a machine-learning model by:
receiving a set of entity data that includes historical prescription data for an entity;
generating a set of training data by labeling each data point in the set of entity data; and
training the machine-learning model, using the set of training data, by mapping the labeled entity data to one or more possible predictions for subsequent prescription executions for the entity;
(a) receiving, by the processing device, data relating to the entity that includes at least one prescription and associated data including a previous execution date of the prescription;
(b) determining, by the processing device and by using predictions output by the trained machine-learning model:
a subsequent execution date; and
at least one action item associated with the entity; and
(c) executing, by the processing device:
the prescription on the subsequent execution date; and
the at least one action item associated with the entity before the subsequent execution date.

2. The method of claim 1, wherein the one or more action items before the execution date comprise at least one action item to inform the entity that the prescription is ready for execution.

3. The method of claim 2, wherein the predictions output by the trained machine-learning model comprise the at least one action item, and wherein the predictions output by the trained machine-learning model comprise an indication that the at least one action item will increase a compliance of the prescription by the entity.

4. The method of claim 2, wherein the one or more action items comprise at least one action item to confirm the execution before the execution date, further comprising determining whether the entity has executed the prescription within a predefined time period determined by the trained machine-learning model, and wherein if the entity has not executed the prescription within the predefined time period, the one or more action items comprises an action item to remind the entity that the execution date has been missed.

5. The method of claim 1, further comprising:
iteratively performing steps a)-c) for a plurality of entities; and
periodically providing a list of the one or more action items for the plurality of entities.

6. The method of claim 5, wherein the one or more action items comprise electronic reminders to a provider to contact an entity.

7. The method of claim 1, further comprising determining one or more suggested execution dates by determining subsequent execution dates for each of the one or more prescriptions and selecting the latest execution date as a suggested execution date.

8. A method comprising:
a) receiving one or more prescriptions, which are associated with an entity, to periodically execute, each of the one or more prescriptions having a subsequent execution date associated therewith, wherein the subsequent execution date is a date when the prescription was last executed incremented by an amount of time determined by a trained machine-learning model;
b) receiving an alignment date selection on a processor for the one or more prescriptions;
c) automatically triggering an execution of the one or more prescriptions on the alignment date; and
d) triggering one or more action items on a processor before and/or after the alignment date, the trained machine-learning model determining the one or more action items.

9. The method of claim 8, wherein the trained machine-learning model is trained by:
receiving a set of entity data that includes historical prescription data for the entity;
generating a set of training data by labeling each data point in the set of entity data; and
training the machine-learning model, using the set of training data, by mapping the labeled entity data to one or more possible predictions for subsequent prescription executions for the entity.

10. The method of claim 8, wherein the one or more action items before and/or after the alignment date comprise at least one action item to inform the entity that the prescriptions are ready for execution.

11. The method of claim 10, wherein the one or more action items comprise at least one action item to confirm the execution before the alignment date, further comprising determining whether the entity has executed the prescription within a predefined time period determined by the machine-learning model, and wherein if the entity has not executed the prescription within the predefined time period, the one or more action items comprises an action item to remind the entity that the prescription has been missed.

12. The method of claim 8, further comprising:
iteratively performing steps a)-d) for a plurality of entities; and
periodically providing a list of the one or more action items for the plurality of entities.

13. The method of claim 8, wherein the one or more action items are electronic reminders to a provider to contact the entity.

14. The method of claim 8, further comprising determining, by the machine-learning model, one or more suggested alignment dates by determining a subsequent execution date for each of the one or more prescriptions and selecting the latest execution date as a suggested alignment date.

15. The method of claim 8, wherein the one or more action items comprise electronic reminders to a pharmacy staff to contact a patient.

16. The method of claim 8, further comprising calculating one or more suggested alignment dates by determining next fill dates for each of the one or more prescriptions and selecting the latest next fill date as a suggested alignment date.

17. The method of claim 8, further comprising determining if any of the one or more prescriptions requires a short fill prior to the alignment date in order to supply a patient with medication until the alignment date.

18. The method of claim 17, further comprising:
if a short fill is required prior to the alignment date, calculating an amount of the short fill; and
filling the amount of the short fill and providing the amount of the short fill to the patient.

19. The method of claim 17, wherein the amount of the short fill is provided to the patient when the patient selects the alignment date.

20. The method of claim 8, further comprising providing the one or more prescriptions to a patient.
